(19) United States
(12) Patent Application Publication
(10) Pub. No.: US 2017/0159129 A1
(43) Pub. Date: Jun. 8, 2017

(54) METHODS FOR DETECTING TELOMERE MAINTENANCE MECHANISMS

(71) Applicant: Thomas Hunt, (US)

(72) Inventors: Thomas Hunt, Saratoga, CA (US); Kelsey Moody, LaFayette, NY (US)

(21) Appl. No.: 15/366,288

(22) Filed: Dec. 1, 2016

Related U.S. Application Data
(60) Provisional application No. 62/262,198, filed on Dec. 2, 2015.

(51) Int. Cl. C12Q 1/68

(52) U.S. Cl. CPC C12Q 1/6886; C12Q 2600/136

(57) ABSTRACT

Presented herein are methods for detecting the activity of alternative lengthening of telomeres, a telomere maintenance mechanism, by measuring relative G-rich and C-rich telomere DNA in a sample. Detection of ALT activity is accomplished with fluorescent probes that recognize G-strand and C-strand telomeric sequences, and fluorescent in-situ hybridization is used to measure the amounts of G-strand and C-strand DNA in a sample cell. The amounts of G-rich and C-rich telomere sequence in the sample cell is determined, relative to the same sequences in a standard, which may be a characterized cell line or a synthetic standard, such as a microbead with telomeric DNA attached.

### CROSS-REFERENCE TO RELATED APPLICATIONS

This application claims benefit of priority pursuant to 35 U.S.C. §119(e) of U.S. provisional patent application No. 62/262,198 entitled “Methods for Detecting Telomere Maintenance Mechanisms,” filed on Dec. 2, 2015, which is hereby incorporated by reference in its entirety.

### FIELD

The present disclosure relates to the field of cancer cell biology and diagnostic devices, methods and compositions for analysis of telomere lengthening.

### BACKGROUND

Most human cells possess a finite capacity for cellular division. After approximately 40-60 doublings in culture, these cells enter replicative senescence and cease to divide, a process colloquially termed the “Hayflick limit”. Induction of senescence is mediated, in part, by the progressive shortening of telomeres. Telomeres are a repetitive nucleotide sequence capping the ends of chromosomal DNA.

Telomere maintenance mechanisms (TMMs) are responsible for maintaining telomere length in select cell populations, such as embryonic stem cells. When strict control of TMM activity is compromised, pre-cancerous cells are able to bypass the Hayflick limit and proliferate unchecked, which can contribute to disease progression. Two TMMs are known to the art. The first is the enzyme telomerase, a ribonucleoprotein that adds telomeric repeats to the 3′ end of telomeres. The second is alternative lengthening of telomeres (ALT), a poorly characterized process that is believed to extend telomere length through homologous recombination.

A study of TMMs in cancerous cell lines found that approximately 85-90% of cancers use telomerase to lengthen their telomeres, whereas the other 10-15% of cancers are dependent upon ALT.

ALT activity is enriched in select cancer subtypes, including osteosarcoma, astrocytoma, glioblastoma multiforme and chondrosarcoma. Interestingly, ALT has also been shown to emerge in telomerase positive cancerous cells that had telomerase deleted. These findings suggest that interventions aimed at treating cancer by specifically targeting the telomerase enzyme may be met with relapse.

Further work is needed to elucidate the mechanism of ALT mediated telomere elongation, and to identify robust inhibitors for therapeutic applications. Progress on these fronts has been significantly impeded by the absence of a sensitive, specific, and high-throughput compatible assay to detect ALT activity.

Disclosed herein is a high throughput method for sensitively and specifically detecting ALT activity in a cell.

### SUMMARY

Disclosed herein are methods of analyzing alternative lengthening of telomeres activity in a sample cell, the methods comprising, permeabilizing the sample cell, and combining it with a nucleic acid probe specific for a G-strand or C-strand telomeric sequence, the amount of nucleic acid probe hybridized to the telomeric sequence in the sample cell is then measured, relative to a standard, and a ratio of G-rich telomeric sequence to C-rich telomeric sequence is calculated to determine if the sample cells has activity for alternative lengthening of telomeres. In many embodiments a G to C ratio of between 0.75 and 1.5 indicates that the sample cell is ALT positive. In many embodiments, the nucleic acid probes include two or more direct repeats of the sequence 5′-CCCTAA-3′, for the C strand, and two or more repeats of the sequence 5′-TTAGGG-3′ for the G-strand, and may also include a fluorescent moiety, for example fluorescein isothiocyanate.

Also disclosed is a method of analyzing alternative lengthening of telomeres activity in a plurality of sample cells, for example a tissue. In this embodiment, the sample cells are combined with a plurality of standards to create a mixture, prior to permeabilizing the mixture and hybridizing nucleic acid probes to the sample cells' telomeric DNA and the standards' telomeric DNA. Flow cytometry is used to measure amounts of the G-strand and C-strand nucleic acid probes in the sample cells, relative to those in the standards, after which a G to C ratio is calculated to help in determining whether the cells are ALT positive or negative. The standards are selected from synthetic sources such as microbeads, for example microbeads containing telomeric DNA, and biological sources such as cells with characteristic telomeric DNA content, for example the T-cell leukemia cell line 1301. In most embodiments, the nucleic acid probes are peptide nucleic acid probes, that include two or more direct repeats of the sequence 5′-CCCTAA-3′ (for the C-strand probe) and for the G-strand probe, two or more repeats of the sequence 5′-TTAGGG-3.′ The probes also include a fluorescein isothiocyanate moiety.

### BRIEF DESCRIPTION OF DRAWINGS

FIGS. 1A-1D: (FIG. 1A) A representative mix of sample cells (SMPL) and 1301 cells analyzed by flow cytometry. These cells have been gated to include only cells (as determined by FSC vs. SSC) and to exclude doublets (as determined by FSC-H by FSC-A). PI stain permits resolution of dividing (DVD) vs. non-dividing (SNG) cells. (FIG. 1B) A histogram of sample cells allows for the measurement of fluorescence. The right peak represents cells. The left peak is debris within the media. (FIG. 1C) A histogram of 1301 cells allows for the measurement of fluorescence. (FIG. 1D) Histograms from probe labeled and probe unlabeled samples are used to obtain quantitative values for fluorescence. The equation for determining relative telomere length is listed. RTL is determined based on C content and G content, then a ratio is created.

FIG. 2: Representative data of relative C to G ratio for select telomerase positive (143B and SJSA-1) and ALT positive (U2OS) cancer cell lines.

FIG. 3: Bar graph showing G to C ratio for various cell lines tested.

FIG. 4 is a spreadsheet of the data represented in FIG. 3.

FIG. 5 is a flow diagram depicting one embodiment of the disclosed method.

### DETAILED DESCRIPTION

### Existing ALT Assays

There are currently two biomarkers that are hallmarks of ALT activity. They are ALT associated promyelocytic leukemia nuclear bodies (APBs) and c-circles.

APBs refer to the co-localization of aggregated promyelocytic leukemia protein (PML) around telomeric sites in ALT positive cells. APBs are detected by labeling two proteins, TRF2, a protein that binds to telomere ends, and PML, with different fluorescent markers. Co-localization is observed using fluorescence microscopy and assaying for co-localization of the two fluorescent markers. The major limitation of the APB assay is that it is labor intensive. It relies heavily on an experienced technician to perform, and it is not easily amenable to a high-throughput screening format.

The C-circle (or G-circle) assay is based upon the existence of partially double stranded extra chromosomal circular telomeric repeats. These circles consist of one complete C-rich or G-rich strand and a second incomplete opposing (complementary) strand. The sequence of the circles is a repeat of six nucleotides; 5′-TTAGGG-3′ for the G-rich strand and 5′-CCCTAA-3′ for the C-rich strand. The origin for C-circles is unclear, but, without wishing to be limited, it is hypothesized they are generated by nucleolytic degradation of the G-rich strand of t-circles, fully double stranded extra chromosomal circular telomeric repeats. It is speculated that t-circles result from the resolution of telomere-loop junctions resulting in free t-circles. C-circles have been found within the nucleus. It has been proposed that the C-circles use rolling circle amplification to lengthen telomeres in ALT-positive cancers or are a byproduct in the process.

A single ALT-positive cell can contain approximately 1,000 C-circles (a complete C-rich strand and incomplete G-rich strand). G-circles are about 100-fold less abundant than C-circles. The abundance of C-circles is substantially elevated in ALT-positive cancers compared to non-cancerous ALT-positive cells. In some cases, this elevation can be as high as 750-fold as compared to telomerase positive cells lines and non-immortalized cells. G-circles are also elevated in ALT positive cancers, but are far less abundant, reducing their utility in identifying ALT-positive cells.

The C-circle assay uses rolling circle amplification to identify and quantify the abundance of c-circles in cells. Briefly, this technique involves isolating DNA from cells, especially circular DNA. The isolated C-circle DNA is then amplified using φ29 DNA polymerase and a radiolabelled nucleotide probe. φ29 polymerase is often used in multiple displacement amplification procedures due to its high fidelity and its ability to produce DNA amplicons greater than 70 kb. Specifically, telomeric C-strand DNA is detected using a ³²P-(TTAGGG)3 probe. The C-circle assay is highly sensitive and specific for ALT activity, but the use of radioactively labeled DNA requires a lengthy development time and special handling, which limits this technique's utility in high-throughput applications. Additionally, the requirement for amplification may lead to skewed results. It is possible to use luminescent markers in replacement of the radioactive ³²P.

Measuring ALT Activity without Radioactivity or Amplification

The present disclosure is directed to an assay for ALT activity that is based on flow cytometry fluorescence in-situ hybridization (flow-FISH) and does not require isolation of c-circles or identification of APBs. Rather, the disclosed method involves measuring the relative amounts of C-rich and G-rich telomeric DNA in a cell, relative to an internal standard. The described use of flow-FISH provides for a sensitive, specific, and accurate method for detecting ALT activity. FISH is a technique that is known to those of skill in the art. Briefly, it involves ‘fixing’ the target cells. Fixation involves immersing the sample in a fixative solution, which terminates any ongoing biochemical reactions and may also increase the mechanical strength and stability of treated samples. Fixing also may help preserve the samples from decay, like autolysis. Next, the ‘fixed’ cells are permeabilized. Permeabilization causes the samples' membranes to open via a permeabilization solution, allowing for passage of solutions into the cells, including markers used to label samples. Permeabilization is followed by labeling of intracellular targets. Where the intracellular targets are DNA target(s), one or more fluorescent nucleic acid probes may be used. In some cases, Peptide-Nucleic Acid (PNA) probes may be used to label samples.

For the presently disclosed techniques, two tests are performed, each using a different PNA probe. The first test uses a probe labeled for identifying G-rich telomeric DNA (e.g. the sequence TTAGGG, which may be repeated three or more times), and the second test uses another probe labeled for identifying C-rich telomeric DNA (CCCTAA, which may be repeated three or more times). Typically, both probes include a fluorescein isothiocyanate FITC moiety for labeling, in other embodiments, moieties other than FITC may be used, for example PE. Such moieties for use in Flow FISH are well known in the art. In some embodiments, the two probes have fluorescent markers that are distinguishable, such that the relative C-rich and G-rich content of each cell can be determined.

Traditionally, FISH labelled cells are subjected to fluorescence microscopy for analysis. However, FISH-labelled samples (e.g. cells) can be analyzed using flow cytometry instead of a fluorescence microscope. This technique has the added benefit that it can be used for quantitating the amount of labelling that is providing net fluorescence for each cell. Thus, not only can the presence of the label in the cell by detected, but the amount of that label in each cell may be quantified. This form of analysis is termed flow-FISH. While unsuitable for many methods, for example where the chromosomal localization of the fluorescent probe is important (i.e. in cytogenetics; the visual observation of chromosomal structure and function), flow-FISH works well with processes involving measurements of net cellular fluorescence. For example, flow-FISH has been successfully used to measure average telomere length within a sample cell population. In the present case, fluorescent probes that are specific for telomeric repeats are used to label the chromosomal DNA of sample cells. Fluorescence from these probes can be measured and compared with fluorescence from a standard, such as the cell line 1301, which is a T-cell leukemia cell line with long heterogeneous telomeres. In some cases, other standards can be used, for example flow cytometry compensation beads (microbeads), wherein the beads are modified with oligonucleotides of telomeric DNA. The use of compensation beads with telomeric oligonucleotides would allow for a more accurate standard, as the oligonucleotides can be set to a more specific (and homogeneous) length than might be found in control/standard cells. In the case of measuring average telomere length, the fluorescence signal of the telomere-specific label for each cell is compared against the fluorescence signal of the standard (cells or beads). The beads consist of 2.8 um MagnaLink magnetic microspheres incorporating 50 kb of Oligo per single microsphere. (The oligo consists of the sequence three direct repeats of the sequence TTAGGG). Samples are stored in PBS with 0.05% sodium azide. This provides for determining, quantitatively, the relative, average telomere length for each cell.

The disclosed methods and compositions are useful in analyzing ALT activity in a cell or tissue. In many embodiments, the cell may be a malignant cell or a non-malignant cell. In most embodiments, the cells and tissues are from a mammal, for example a human. Tissue may be solid tissue or tumor, or may be a liquid biopsy (for example blood, lymph, bone marrow, or a cell derived from blood, lymph, or bone marrow).

While multiple embodiments of the present methods and systems are described, still other embodiments of the present disclosure will become apparent to those skilled in the art from these descriptions. As will be apparent, the invention is capable of modifications in various obvious aspects, all without departing from the spirit and scope of the present invention. Accordingly, the description is to be regarded as illustrative in nature and not restrictive.

### Quantitative Analysis of ALT Activity by Flow FISH

The present disclosure is directed to a novel improvement of flow-FISH that has been adapted for quantifying ALT activity in a cell. Briefly, cells to be analyzed are first mixed with an internal standard for telomeric DNA content. In one embodiment, the 1301 cell line, a T-cell leukemia cell line with long homogeneous telomeres is used. In other embodiments, flow cytometry microbeads with telomeric oligonucleotides can also be used to provide a standard. In one embodiment, wherein the probes have the same fluorescent marker, the sample+standard mixtures are first divided into three containers: one for measuring C-rich telomeric DNA; one for measuring G-rich telomeric DNA; and one with no probe at all added. The sample+standard mixtures are then fixed and permeabilized. This is done by incubating samples in a hybridization buffer, at a concentration of roughly 5,800 cells per μL (in some embodiments the concentration may range from 1,000 to 10,000 cells per μL) to create a cell+hybridization buffer mixture. One exemplary buffer may be found as detailed in “Flow cytometry and FISH to measure the average length of telomeres (flow FISH)” (Nature Protocols 1, p. 2365-2376, Baerlocher et al. 2006), which is incorporated by reference in its entirety. The appropriate PNA probe is then combined with the hybridization buffer+cell mixture to create a probe+cell+buffer sample. The probe+cell+buffer samples are then incubated by being placed in or on a heating block set to 82° C. for 10 minutes. While the samples are being fixed and permeabilized, the Peptide Nucleic Acid (PNA) probe is also binding to the target telomeric DNA. The two PNA probes bind to C-rich telomeric DNA (in which the PNA comprises three (3) repeats of the sequence TTAGGG), or G-rich telomeric DNA (in which the PNA comprises three (3) repeats of the sequence CCCTAA). In many embodiments, both PNA probes are labeled with a FITC moiety. In other embodiments, the PNA probes may include other appropriate labels. In some embodiments, the labels have the same fluorescence and stability characteristics (e.g. FITC). In other embodiments, the labels for the two probes may be different and have different fluorescence and stability characteristics. In general, PNAs are artificially synthesized nucleotides with a higher binding strength, making it useful for diagnostic assays. After incubation, cells are washed in flow cytometry staining buffer and then analyzed by flow cytometry.

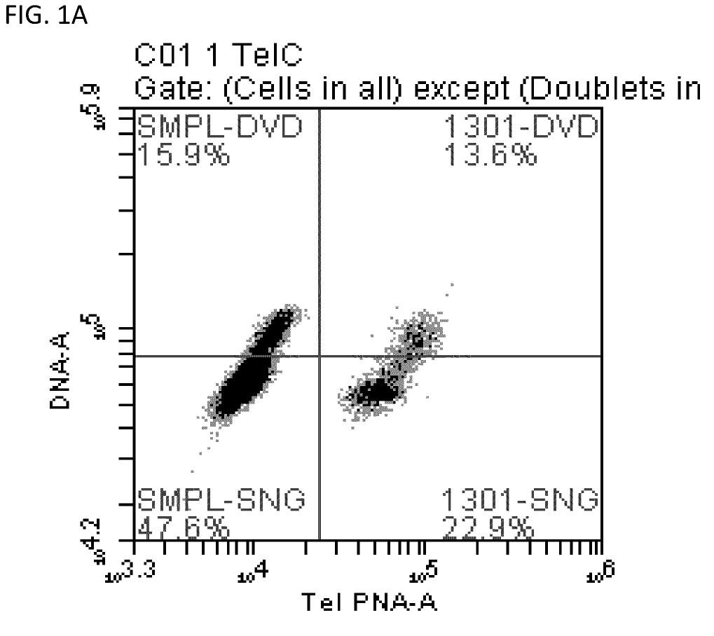

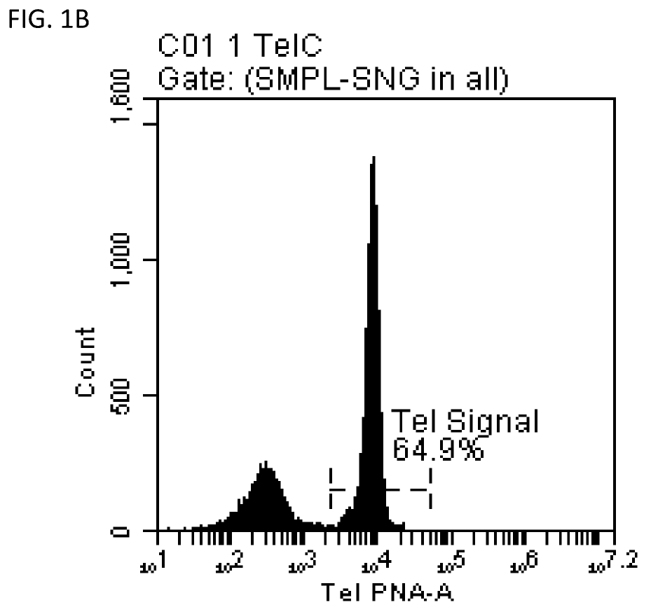

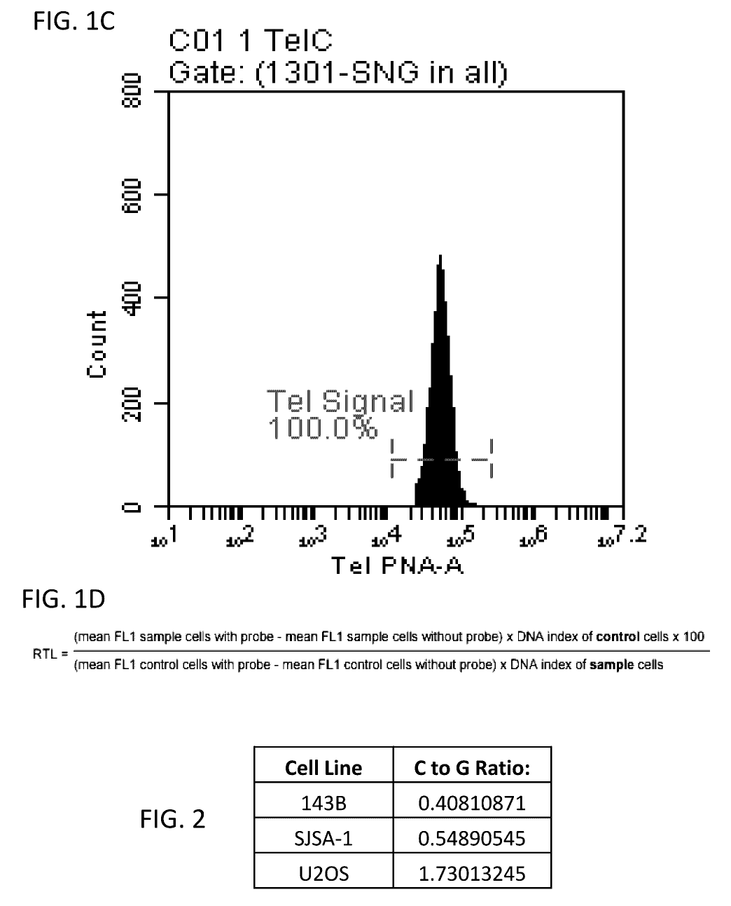

Relative C-strand content and relative G-strand content of the sample cells is determined, and a ratio of G content to C content calculated. One exemplary equation for determining these values is shown in FIG. 1D. First, the mean fluorescence of the probes is measured for both sample cells and standard. In most embodiments, the standard is a cell with a characteristic telomere content, for example in one embodiment, 1301 cells that have a high telomere content can be used. In other embodiments, mircobeads with attached telomeres, or a cell with homozygous or long telomeres may be used. In most embodiments, the standard may further include a fluorescent marker that is different from that used to label the Tel G and Tel C probes. The fluorescent marker in the standard allows for identification of the internal standard and separation of its Tel G and Tel C probe fluorescence. In many embodiments, the internal standard may include a red fluorescent label, for example red fluorescent protein (RFP). In other embodiments, other suitable internal standards may be selected, wherein the marker of the internal standard is other than RFP. Including a separate fluorescent marker with the standard allows, for example, the mean fluorescence of the Tel G probes in the sample cells to be compared to the mean fluorescence of the Tel G probes in the internal standard cells.

The mean fluorescence of samples with Tel G probes and Tel C probes is measured, and the amount of background fluorescence (determined by measuring cells without probes) deducted from these values to create a corrected mean fluorescence for the sample cells. Next, the mean fluorescence of standards with Tel G probes and Tel C probes is measured, and, again, the amount of background fluorescence (as determined by measuring the no probe samples) is deducted. The corrected mean fluorescence values may be further adjusted where the DNA index (ploidy, or chromosomal copy number) of the sample is different from that of the standard (again, see the RTL equation at FIG. 1D). In some embodiments, the adjusted mean fluorescence of the sample may be multiplied by the DNA index, or ploidy value, of the internal standard, and the mean fluorescence of the standard may be multiplied by the DNA index, or ploidy value, of the sample. For example, some cells, such as 1301 cells are tetraploidy, so the DNA index for 1301 cells is 2, but most cells are diploid, so the DNA index for most cells is 1. Many osteosarcomas, such as those used below, are diploid and multiplying by this DNA index, ‘1,’ will leave the adjusted mean fluorescence value unchanged.

The corrected mean fluorescence of the sample is then divided by the mean fluorescence of the standard to produce a relative fluorescence value; for example the Tel G mean fluorescence of the sample is divided by the Tel G mean fluorescence of the standard (both values having been adjusted for differences in DNA index) to produce a relative Tel G value (RTL; see FIG. 1D). Finally, the relative Tel C value is divided by the relative Tel G value to create a G to C ratio.

ALT activity is determined based on the G to C ratio. If the G to C ratio is greater than 0.8, the sample may referred to as being positive for ALT activity. In some embodiments, the G to C ratio of an ALT positive cell line may be from about 0.75 to about 1.7. In some embodiments, the G to C ratio of an ALT positive cell line may be greater than about 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.00, 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, or 1.19, and less than about 1.20, 1.19, 1.18, 1.17, 1.16, 1.15, 1.14, 1.13, 1.12, 1.11, 1.10, 1.09, 1.08, 1.07, 1.06, 1.05, 1.04, 1.03, 1.02, 1.01, 1.00, 0.99, 0.98, 0.97, 0.96, 0.95, 0.94, 0.93, 0.92, 0.91, 0.90, 0.89, 0.88, 0.87, 0.86, 0.85, 0.84, 0.83, 0.82, 0.81, 0.80, 0.79, 0.78, 0.77, or 0.76. If the G to C ratio is less than 0.7, the sample may be referred to as being negative for ALT activity. In some embodiments, if the G to C ratio is less than about 0.75, 0.70, 0.65, 0.60, 0.55, 0.50, 0.45, or 0.40 the cell may be referred to as ALT negative.

This disclosure is superior to current ALT activity assays in several ways. First, it is faster, requiring few hands-on steps during preparation and shorter incubations. Second, the method can be adopted for use in an automation friendly microplate format, making it suitable for high-throughput screening applications. Lastly, it eliminates the need for radioactive isotope labeling, which supports broader use in clinical diagnostics and routine laboratory testing.

### EXAMPLES

### Example 1—Diagnostic Application in Clinical Laboratory

1×10⁵ primary glioblastoma multiforme cells (ATCC CRL-1690 cells) are grown in Eagle's Minimum essential Medium with 10% FBS. Viable cells were enumerated by trypan blue exclusion using a hemocytometer. The multiforme cells were mixed 1:1 with the T-cell leukemia cell line 1301 (available from Sigma-Aldrich), expressing RFP, red fluorescent protein. 1301 cells had been transformed with RFP to distinguish them from sample cells in the flow cytometer. The RFP could be distinguished from the Tel G and Tel C PNA probes, which were detected on fluorescence channel 1, FL1 (Absorbance 492 nm; Emission 518 nm) while RFP is detected in FL3 (Absorbance 488 nm Emission 532 nm). Cell mixtures were then separated into two 1.5 mL Eppendorf tubes (marked ‘A’ and ‘B’), washed with PBS obtained from cellgro, then resuspended in 250 uL permeabilization/fixation buffer or hybridization mixture. The Hybridization mixture consists of 6.05 g Tris in 50 mL H2O in which HCl was added until the pH reached 7.1. Then 8.37 mL 10% BSA, 1.67 mL 1M NaCl and 62.7 mL deionized formamide was added, stirred and stored at −20° C. until needed. All materials for the solution were acquired from sigma Aldrich. In tube A, TeIC-FITC (PNA Bio cat F1009, manufactured by PNA bio sequence 5′-CCCTAA-3′ repeated three times, excitation spectrum: 492 nm emission spectrum: 518 nm.) was added. In tube B, TeIG-FITC (PNA Bio cat F1010, manufactured by PNA bio sequence 5′-TTAGGG-3′ repeated three times, excitation spectrum: 492 nm emission spectrum: 518 nm) was added. The cells were resuspended and mixed cells were incubated for 14.5 hours in the dark at room temperature, then washed 3 times in flow cytometry staining buffer (FCSB) manufactured by ebioscience Cat no. 00-4222-26. Cells were then resuspended in 1 mL FCSB and submitted to flow cytometry for analysis.

For flow cytometry, a BD Accuri C6 flow cytometer was used. The flow cytometer was set for logarithmic scale FL1-H for probe fluorescence and logarithmic scale FL3-H for RFP fluorescence. Side-scattered light (SSC) vs forward-scattered light (FSC) and RFP fluorescence were used to establish gates for the sample and for the standard (1301) cells. 1301 cells and samples cells treated separately were analyzed to determine where each cell line would be located, allowing the setting of gates for the two cell lines. Data was analyzed to determine the amount of Tel C content and Tel G content of the sample cells relative to the 1301 cells. If G to C content was greater than 0.8, the result was labelled as ALT positive.

### Example 2—Mechanism Study in Research Laboratory

The ALT positive cell line USOS was cultured in McCoy's 5A containing 10% FBS at 37 C in 5% CO2. Expression of Shelterin complex proteins TRF1, TRF2, POT1, RAP1, TIN2, or TPP1 was knocked down by incubation with siRNA lentivirus specific to each target in the presence of polybrene. Knockout cells were selected by G418, and knockdown was confirmed by western blot. For each knockout, 2×10⁵ cells were enumerated by trypan blue exclusion using a Countess Automated Cell Counter and mixed 1:1 with fresh bovine thymocytes. Samples were separated into two 1.5 mL Eppendorf tubes. Cells were washed with HBSS and treated with TeIC-PE or TeIG-PE. Cells were then incubated for 18 hours in the dark at room temperature. After incubation, cells were incubated in HBSS for 1 hour, pelleted, then re-suspended in 1 mL HBSS and analyzed by a BD Accuri C6 flow cytometer using logarithmic scale FL2-PE for probe fluorescence. SSC vs. FSC were used to establish gates for the sample and for the bovine thymocytes. Data was analyzed to determine C to G content in the sample cells relative to the bovine cells. C to G content was measured. Each sample was assayed in triplicate, and p-values were calculated to determine if gene knockdown led to a reduction in ALT activity in ALT positive cells. Cells where C to G content was less than 1 were considered to be ALT negative.

### Example 3—Small Molecule Drug Screen Application

The ALT positive cell line Saos-2 was grown to confluency in a 150 mm tissue culture treated dish, then incubated in McCoy's 5A containing 10 ug/mL mitomycin C for 2 hours to achieve mitotic inactivation. Cells were then detached by incubating in 0.25% trypsin containing EDTA for 5 minutes, resuspended in DMEM containing 10% FBS, and enumerated using a Burker counting chamber. Cells were diluted to 3.5×105 cells/mL, and 100 uL were added to all wells of a 96-well tissue culture treated plate. The first column was untreated. For the remaining 88 wells, two wells were treated with one of 44 different small molecules from the Ichor ChemFactor™ compound library for 48 hours. After treatment, the plate was washed three times in ISP wash buffer (Biolegend cat 421002). Each well set was treated with either TeIC-FITC or TeIG-FITC, then incubated in the dark for 10 hours at room temperature. Cells were washed three times with ISP wash buffer and re-suspended in 100 uL of PBS. Cells were analyzed by Molecular Devices Gemini XS microplate reader using Ex488/Em533. C to G content of treated cell pairs was compared to baseline control. Treatments resulting in a large reduction of C to G ratio compared to baseline were selected for further evaluation.

### Example 4—the Inhibition of ALT Activity in ALT+ Cell Lines

The ALT+Cell Line U2OS, Saos-2 and G-292 (ALT+Cell lines) were grown to confluency in a 150 mm tissue culture treated dish in McCoy's 5A 10% FBS along with 143B, SJSA-1 and HOS cell lines which use telomerase to lengthen telomeres. Another population of these cell lines was grown to confluency and incubated with 3 mM HU in 25 mL of McCoy's 5A 10% FBS. The cells were incubated for 48 Hours and washed 3 times in PBS. Cells were then placed into tubes with 1301 cells to act as a standard and samples were centrifuged. Samples were given Hybridization mixture as detailed in Example 1, containing PNA probe, either for labeling C rich telomeric DNA, G rich telomeric DNA, or no PNA probe at all. The samples were allowed to incubate for 14.5 hours and washed twice with Flow Cytometry staining buffer. Samples were then resuspended in Flow Cytometry staining buffer and measured with flow cytometry. The mean FI1 of each cell line was used to find the relative telomere length, as specified in previous examples. The relative telomere length of C content to G content was compared to get a G to C Ratio ALT+Cell lines were found to be above 0.8 while Telomerase+ cell lines were found below 0.8. The ALT+cell lines that were given hydroxyurea were found to have a ratio below 0.8 indicating a decrease in ALT activity.

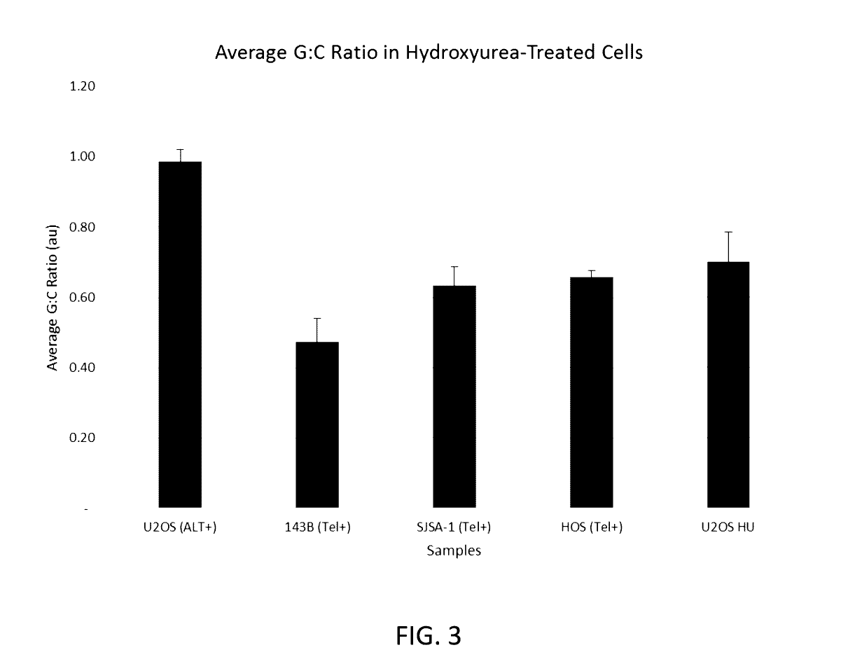

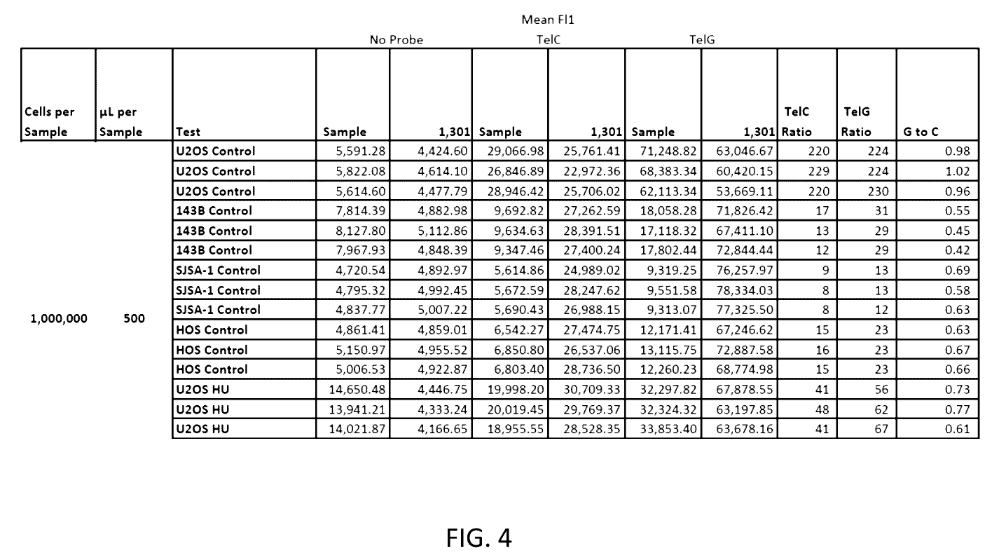

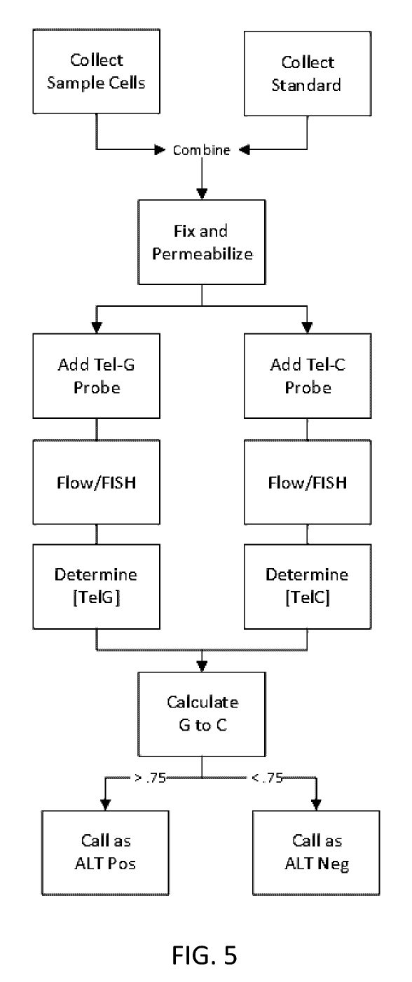

Results of these experiments are shown at FIG. 3, in bar graph form, and FIG. 4 as a spreadsheet of data.

All references disclosed herein, whether patent or non-patent, are hereby incorporated by reference as if each was included at its citation, in its entirety. In case of conflict between reference and specification, the present specification, including definitions, will control.

Although the present disclosure has been described with a certain degree of particularity, it is understood the disclosure has been made by way of example, and changes in detail or structure may be made without departing from the spirit of the disclosure as defined in the appended claims.

We claim:

1. A method of analyzing alternative lengthening of telomeres activity in a sample cell comprising:
permeabilizing the sample cell;
combining the permeabilized sample cell with a C-strand nucleic acid probe;
measuring the amount of the C-strand nucleic acid probe in the permeabilized sample cell;
combining a permeabilized sample cell with a G-strand nucleic acid probe;
measuring the amount of the G-strand nucleic acid probe in the permeabilized sample cell;
dividing the amount of the C-strand probe by the amount of G-strand probe to determine a ratio of the C-strand probe to the G-strand probe; thereby
analyzing the activity of alternative lengthening of telomeres activity in a sample cell.

2. The method of claim 1, wherein C-strand probe includes two or more repeats of the sequence 5′-CCCTAA-3′.

3. The method of claim 1, wherein G-strand probe includes two or more repeats of the sequence 5′-TTAGGG-3′.

4. The method of claim 1, wherein the G-strand probe and the C-strand probe include a fluorescent moiety.

5. The method of claim 4, wherein the G-strand and C-strand probes are peptide nucleic acid compounds.

6. The method of claim 5, wherein the fluorescent moiety is fluorescein isothiocyanate.

7. The method of claim 1, wherein if the G to C ratio is between about 0.75 and about 1.5, the sample cell is referred to as ALT positive.

8. A method of analyzing alternative lengthening of telomeres activity in a plurality of sample cells comprising:
combining the sample cells with a plurality of standards to create a mixture;
permeabilizing the mixture of sample cells and standards;
combining at least one permeabilized sample cell with a C-strand nucleic acid probe;
combining at least one permeabilized sample cell with a G-strand nucleic acid probe;
combining at least one standard with C-strand nucleic acid probe;
combining at least one standard with G-strand nucleic acid probe;
allowing the nucleic acid probes to hybridize to a complementary nucleic acid sequence;
measuring the amount of the nucleic acid probe hybridized to sample cells and standards by flow cytometry;
dividing the amount of the C-strand nucleic acid probe in the sample cells by the amount of C-strand nucleic acid probe in the standard to produce a relative C-strand value;
dividing the amount of the G-strand nucleic acid probe in the sample cells by the amount of G-strand nucleic acid probe in the standard to produce a relative G-strand value;
dividing the relative C-strand value by the relative G-strand value to produce a G to C ratio, thereby
analyzing the activity of alternative lengthening of telomeres activity in a sample cell.

9. The method of claim 8, wherein the standard is selected from a cell and a microbead.

10. The method of claim 9, wherein the cell is a T-cell leukemia cell, 1301.

11. The method of claim 10, wherein the microbead includes telomeric DNA.

12. The method of claim 8, wherein nucleic acid probes are peptide nucleic acid probes.

13. The method of claim 12, wherein the C-strand probe includes two or more repeats of the sequence 5′-CCCTAA-3′ and the G-strand probe includes two or more repeats of the sequence 5′-TTAGGG-3.′

14. The method of claim 13, wherein the probes include a fluorescein isothiocyanate moiety.

15. The method of claim 1, wherein if the G to C ratio is between about 0.75 and about 1.5, the sample cell is referred to as ALT positive.

16. A method of analyzing alternative lengthening of telomeres activity in a tissue comprising:
combining a plurality of cells from the tissue with a standard to create a sample mixture;
permeabilizing sample mixture;
combining the permeabilized sample mixture with a fluorescent C-strand nucleic acid probe and fluorescent G-strand nucleic acid probe in a hybridization buffer;
allowing the nucleic acid probes to hybridize to complementary sequences within the sample cell and in or on the standard;
measuring the amount of the each nucleic acid probe in the cells from the tissue relative the standard;
dividing the relative amount value of the C-strand probe by the relative amount value of the G-strand probe to determine a ratio of the G-strand probe to the C-strand probe in the cells from the tissue; thereby
analyzing the activity of alternative lengthening of telomeres activity in a tissue.

17. The method of claim 16, wherein the C-strand and G-strand nucleic acid probes are fluorescent probes, comprising the sequences CCCTAA and TTAGGG, respectively.

18. The method of claim 16, wherein the amount of C-strand and G-strand probe is measured by flow cytometry.

19. The method of claim 16, wherein the sample is a plurality of cells selected from cell lines having well-characterized telomeres, long homogeneous telomeres, ALT activity, and combinations thereof.

20. The method of claim 19, wherein the tissue is treated with therapeutic protocols that do not target telomerase.
